(19) United States
(12) Patent Application Publication
(10) Pub. No.: US 2024/0290462 A1
(43) Pub. Date: Aug. 29, 2024

(54) Generating Multi-Sensory Content based on User State

(71) Applicant: Microsoft Technology Licensing, LLC, Redmond, WA (US)

(72) Inventors: Judith AMORES FERNANDEZ, Cambridge, MA (US); Mary Patricia CZERWINSKI, Kirkland, WA (US); Javier HERNANDEZ RIVERA, Somerville, MA (US)

(73) Assignee: Microsoft Technology Licensing, LLC, Redmond, WA (US)

(21) Appl. No.: 18/115,501

(22) Filed: Feb. 28, 2023

(51) Int. Cl. G16H 20/70; G06N 20/00

(52) U.S. Cl. CPC G16H 20/70; G06N 20/00

(57) ABSTRACT

A technique for providing multi-sensory content receives input information that expresses a physiological state and experienced emotional state of a user. The technique generates prompt information that describes at least an objective of guidance to be delivered and the input information. The technique maps the prompt information to output information using a pattern completion component. The output information contains control instructions for controlling an output system to deliver the guidance via generated content. In some implementations, the pattern completion component is a machine-trained pattern completion model. In some implementations, a reward-driven machine-trained model further processes the input information and/or the output information. The reward-driven machine-trained model is trained by reinforcement learning to promote the objective of the guidance. In other implementations, the reward-driven machine-trained model operates by itself, without the pattern completion component.

### BACKGROUND

There have been attempts to integrate computing technology into training sessions. In some cases, the objective of a training session is to achieve a specified therapeutic goal. But the solutions that have been proposed are sometimes not scalable. That is, the solutions are developed to serve narrow objectives of specific training environments and cannot easily be modified to serve other training environments. Further, the individual solutions are sometimes labor-intensive and resource-intensive to develop, maintain, and use. Finally, the solutions may fail to adequately engage a user's attention, which negatively impacts their ability to achieve a stated goal of a training session.

### SUMMARY

A computer-implemented technique is described herein for providing personalized multi-sensory content based on input information that expresses a user's current state, including information that expresses a physiological state of the user and/or an experienced emotional state of the user. The technique generates prompt information that describes the input information and an objective of guidance to be delivered. The technique then uses a pattern completion component to map the prompt information to output information. The output information contains control instructions for directing an output system to deliver the guidance via the multi-sensory content.

In some implementations, the objective expressed in the prompt information is a therapeutic goal of the guidance. In some examples, the therapeutic goal is: (a) reduction of stress; or (b) meditation; or (c) inducement of sleep; or (d) promoting any of attentiveness, mindfulness, and focus (and/or reducing sleep onset); or (e) control of a specified emotion or compulsion; or (f) management of memory; or (g) ability to complete a task within a specified environment; or (h) enhancement of productivity; or (i) any combination thereof.

In some implementations, the physiological state of the user expresses any of: (a) one or more vital signs; or (b) brain activity; or (c) electrodermal activity; or (d) body movement; or (e) one or more eye-related characteristics; or (f) one or more voice-related characteristics; or (f) any combination thereof. In some implementations, the technique allows the user to self-report his or her current emotional state, e.g., by inputting “anxious” when the user feels anxious.

In some implementations, the pattern completion component is a machine-trained model that maps text-based input information to text-based output information. In some cases, at least some of the text-based output information includes instructions for controlling one or more sensing devices in the form of commentary, commands, numerical values (associated with respective settings), etc., or any combination thereof. In some examples, the machine-trained model is a transformer-based model that includes an attention mechanism.

In some implementations, the output system includes: (a) an audio output system for delivering audio content; or (b) a visual output system for delivering visual content; (c) a lighting system for modifying lighting in the user's environment; or (d) an odor output system for delivering scents; or (e) a haptic output system for delivering a tactile experience; or (f) an HVAC system for controlling heating, cooling, and/or ventilation or (g) a workflow-modifying system; or (h) any combination thereof. In some cases, the audio content includes a narrative generated by the pattern completion component that aims to advance the therapeutic goal of the guidance.

In some implementations, the visual output system uses another machine-trained model to synthesize visual content based on the output information generated by the pattern completion component. An environment can present the synthesized visual content together with other sensory experiences delivered by other output systems (including the audio output system, the odor output system, the haptic output system, etc.).

In some implementations, another machine-trained model further processes the input information and/or the output information. This machine-trained model is trained by reinforcement learning to promote the objective of the guidance, e.g., by promoting particular content that advances the objective, and penalizing other content that impedes achievement of the objective. In other implementations, the machine-trained model operates by itself, without the pattern completion component.

Overall, the technique provides a flexible tool for serving many different training objectives using a common processing model. The tool thereby eliminates or reduces the time-intensive and resource-intensive prior practice of developing and maintaining standalone systems that serve specific training objectives. Further, the tool is user-friendly because a user is able to easily apply it to a specific training problem. That is, the user need only articulate a goal of training and his or her current emotional state to successfully set up the tool for a specific training environment. In addition, or alternatively, the multi-sensory content is guided by inferences drawn from other sources, including sources that provide context information. The context information includes information regarding a task that the user is performing, information regarding calendar events, information from various environment-sensing devices, etc. Further, the technique provides a sensory experience that is engineered to capture and hold the attention of a user, which increases the chances that training will prove successful.

This Summary is provided to introduce a selection of concepts in a simplified form; these concepts are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 shows a computing system for providing multi-sensory content to achieve a training objective.

FIG. 2 shows an example of multi-sensory content delivered by the computing system of FIG. 1.

FIG. 3 shows an example of a prompt generating component, which is one component of the computing system of FIG. 1.

FIG. 4 shows an example of seed prompt information generated by the prompt-generating component of FIG. 3.

FIG. 5 shows an example of another computing system for providing multi-sensory content that incorporates the use of reinforcement learning.

FIG. 6 shows one example of a machine-trained pattern completion model, which is another component of the computing systems of FIGS. 1 and 5.

FIG. 7 shows one example of a machine-trained image synthesis model, which is another component of the computing systems of FIGS. 1 and 5.

FIG. 8 shows a process that provides an overview of one manner of operation of the computing systems of FIGS. 1 and 5.

FIG. 9 shows a process that provides an overview of one manner of operation of the computing system of FIG. 5.

FIG. 10 shows computing equipment that, in some implementations, is used to implement the computing systems of FIGS. 1 and 5.

FIG. 11 shows an illustrative type of computing system that, in some implementations, is used to implement any aspect of the features shown in the foregoing drawings.

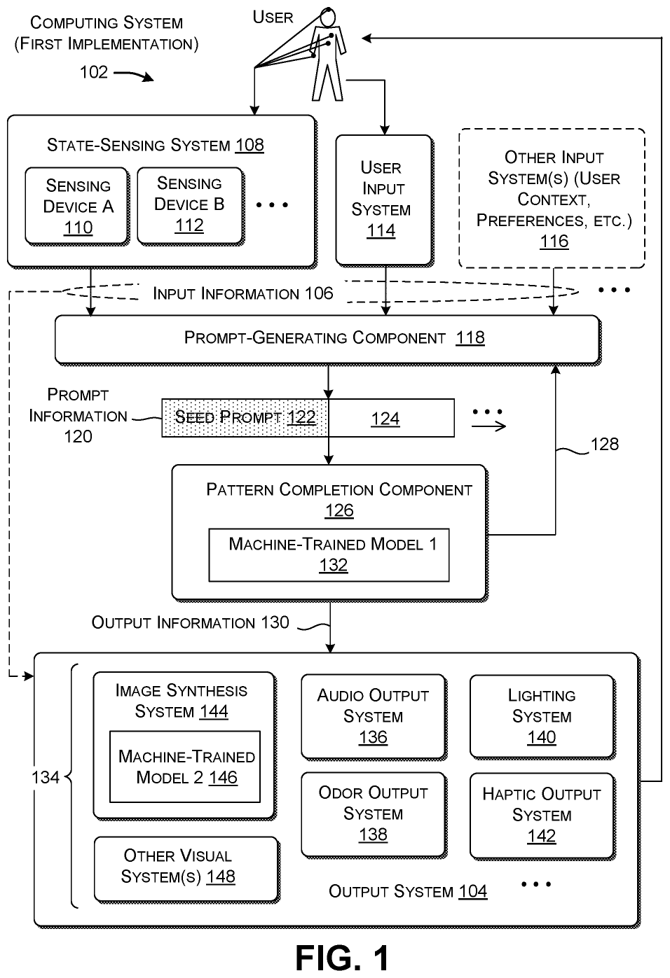

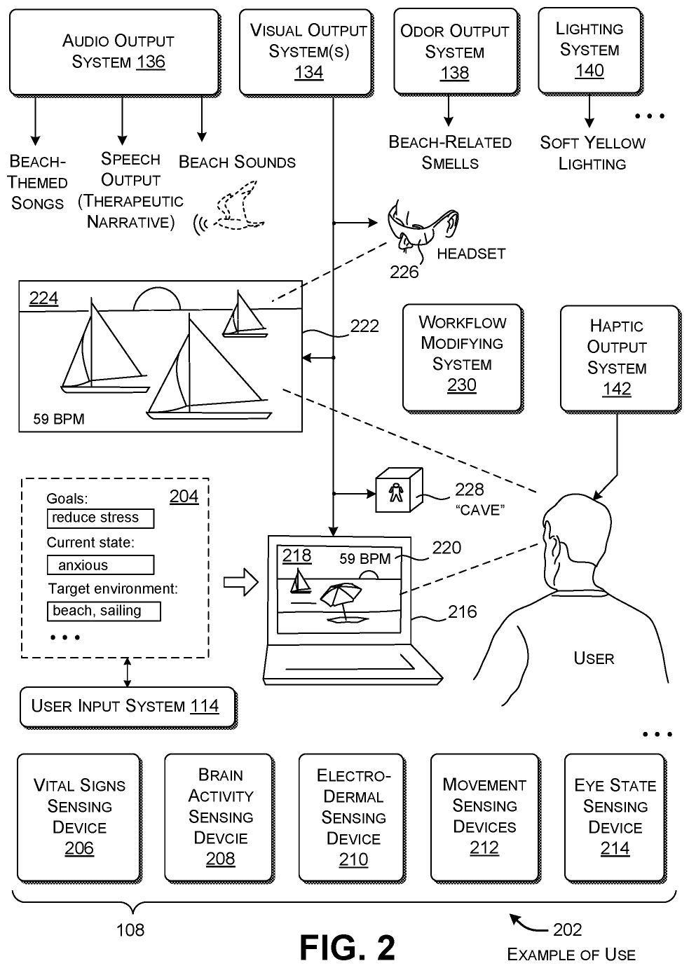

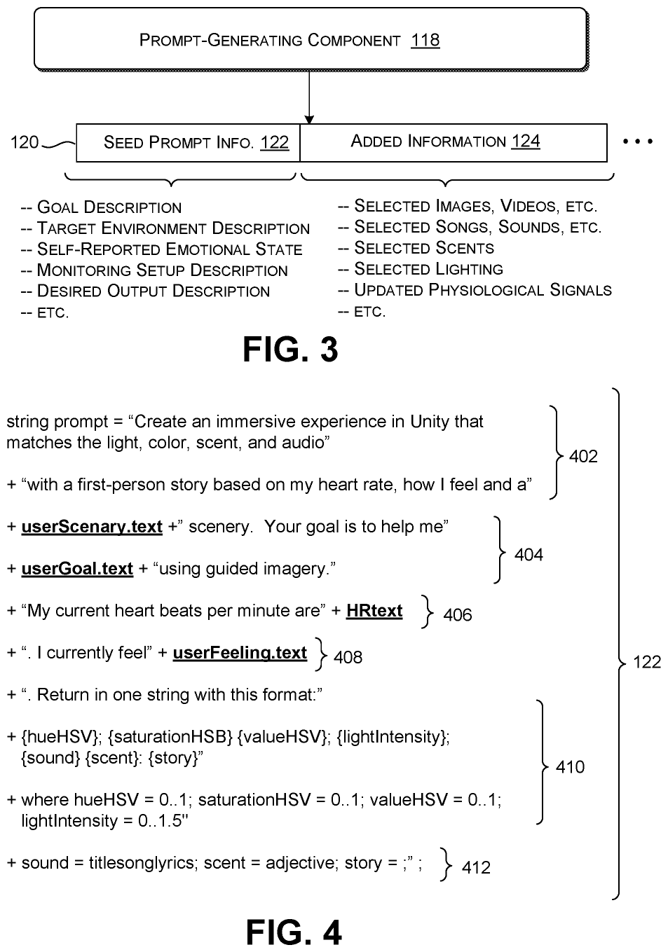

The same numbers are used throughout the disclosure and figures to reference like components and features. Series 100 numbers refer to features originally found in FIG. 1, series 200 numbers refer to features originally found in FIG. 2, series 300 numbers refer to features originally found in FIG. 3, and so on.

### DETAILED DESCRIPTION

FIG. 1 shows an illustrative computing system 102 for delivering multi-sensory content to a user via an output system 104. As will be described in greater detail below, the computing system 102 is implemented by one or more computing devices. In some cases, an entirety of the computing system 102 is implemented by a local computing system. In other cases, aspects of the computing systems 102 are distributed between local and remote computing resources.

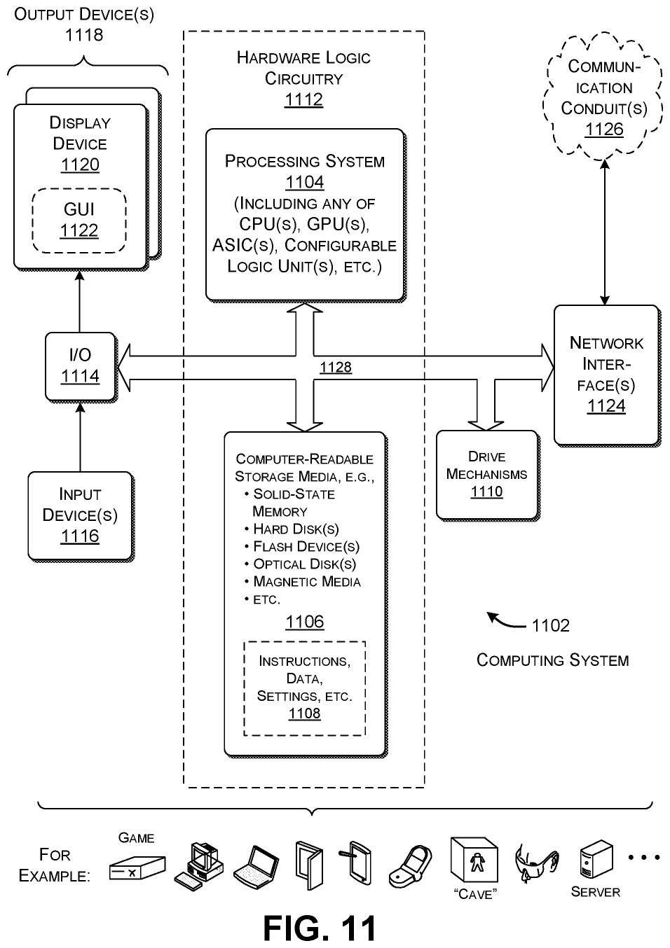

By way of terminology, a “machine-trained model” refers to computer-implemented logic for executing a task using machine-trained weights that are produced in a training operation. “Weights” is shorthand reference to parameter values. In some contexts, terms such as “component,” “module,” “engine,” and “tool” refer to parts of computer-based technology that perform respective functions. FIGS. 10 and 11, described below, provide examples of illustrative computing equipment for performing these functions.

The computing system 102 receives input information 106 from various input systems. For instance, the input information 106 expresses one or more aspects of a current physiological state of the user, as captured by a state-sensing system 108. The sensing system 108 includes one or more sensing devices (110, 112, . . . ). Without limitation, the physiological state of the user expresses: (a) one or more vital signs (e.g., as captured by sensing devices that measure heart beat rate, blood pressure, respiratory rate, and/or temperature); (b) one or more brain activity states (e.g., as captured by an electroencephalogram sensing device); (c) one or more eye-related states (e.g., as captured by a sensing device that measures pupil dilation or a sensing device that determines direction of gaze); (d) one or more skin response states (e.g., as captured by any type of electrodermal sensing device); (e) any body movements; (f) one or more voice-related states; or any combination thereof.

The input information 106 also includes information items explicitly specified by the user via a user input system 114. One such information item describes a type of environment to be depicted in the multi-sensory content. Examples of environments include: (a) beach-related settings; (b) mountain-related settings; (c) forest-related settings; (d) urban settings; (e) workplace-related settings (such a surgeon's operative room); (f) any historical settings, and so on. In some cases, a historical setting corresponds to a historical setting of shared interest (e.g., Brooklyn, New York in the 1970s). In other cases, a historical setting corresponds to a personal environment of a particular user (e.g., depicting a user's own home environment in Brooklyn, New York in the 1970s, with all of its accompanying sensory stimuli and reminders of the people associated with that prior environment). The computing system 102 provides an interface (not shown) that allows the particular user to specify the private setting, for example by providing a textual description of the private setting, keywords, images, videos, audio recordings, links to news stories, etc. Alternatively, or in addition, the computing system 102 creates the private setting on behalf of the particular user, with the user's explicit authorization. The computing system 102 is also able to draw from one or more knowledge bases, search engines, question-answering services, chat bots, etc. in constructing a setting.

Another information item describes the user's self-reported emotional state. Another information item describes the purpose of the training. Without limitation, some illustrative goals of training include: (a) reduction of stress; or (b) meditation; or (c) inducement of sleep; or (d) inducement of any of attentiveness, mindfulness, and focus (and/or reducing sleep onset); or (e) control of a specified emotion or compulsion; or (f) management of memory; or (g) ability to complete a task within a specified environment; or (h) enhancement of productivity; or (i) any combination thereof. Different implementations of the user input system 114 solicit information items from the user in different respective ways. In some examples, the user input system 114 collects the information items from the user via a user interface page, for instance, as free-form text entries and/or as selections within drop-down menus or the like.

In some examples, the input information 106 includes other information from any other sources 116. For instance, in some implementations, the input information 106 includes an information item that specifies a current location of the user obtained from any location-determining device or service. One such location-determining device is a global positioning system (GPS) device. The location of the user is also inferable based on the user's proximity to beacons and cell towers having known positions. In addition, or alternatively, the input information 106 specifies the current time and/or the available time to perform a task. In addition, or alternatively, the input information 106 specifies the nature of the user's current physical environment, e.g., by specifying whether the user is interacting with the computing system 102 at home or at the user's workplace. In addition, or alternatively, the input information 106 includes any data obtained from any environmental sensor or detection device, including any weather sensors, any traffic sensors, any news feed information, and so on. In addition, or alternatively, the input information 106 expresses any characteristics of the user, including the user's age, location, interests, media preferences, etc. Individual users are given the ability to authorize (or prohibit) the collection of this kind of information, and to specify the conditions of the use of this information.

In addition, or alternatively, the input information 106 expresses any information regarding a task that a user is currently performing, or has recently performed. In addition, or alternatively, the input information 106 includes information extracted from a personal or shared calendar (with the explicit authorization of the user), e.g., pertaining to events that a user is scheduled to perform or other planned occurrences that may have an impact on the user. In addition, or alternatively, the input information 106 includes information from an inference mechanism that predicts a user's current physiological and/or emotional state based on behavior exhibited by the user and/or other context-based signals (such as calendar events). For example, the input information 106 may include a prediction that the user is likely stressed based on the number of meetings that the user has scheduled, and/or the number of email messages that the user has yet to read, and/or the number of impending deadlines to which the user is subject, and so on. In some implementations, the computing system 102 implements an inference mechanism that is capable of performing a predictive function using a machine-trained model of any type, examples of which are described below.

A prompt-generating component 118 generates prompt information 120 based on the input information 106. In some implementations, the prompt-generating component 118 performs this task by assembling the parts of the input information 106 and other pre-generated content into a series of textual tokens. Each token expresses a word or a part of the word. For instance, part of the prompt information 120 includes the pre-generated text fragment “My current heart beats per minute are.” Assume that a heart rate sensing device reveals that the user's current heart rate is 68 beats per minutes. The prompt-generating component 118 appends the text “68 bpm” to the end of the pre-generated text fragment, such that the text fragment now reads, “My current heart beats per minute are 68 bpm.”

More specifically, in some examples, the computing system 102 operates in multiple passes in an autoregressive manner. The prompt-generating component 118 creates seed prompt information 122 on a first pass. The seed prompt information 122 describes the principal aspects of the training to be delivered to the user, e.g., by specifying the purpose of the training, the type of environment to be depicted in the multi-sensory content, and the user's current physiological and emotional states. The prompt-generating component 118 appends added information 124 to the end of the existing prompt information 120 upon each successive pass. The added information 124 expresses the multi-sensory content that has been delivered to the user, the user's updated physiological and/or emotional state, and so on.

A pattern completion component 126 analyzes the prompt information 120, and generates a prediction of text that is likely to follow the prompt information 120. For instance, assume that the seed prompt information 122 is composed of a series of text tokens, S₁, S₂, S₃, . . . , S_{N}. The pattern completion component 126 successively determines a series of one or more text tokens (A₁, A₂, A₃, . . . , A_{K}) that are likely to follow the seed prompt information 122 over multiple successive passes, each time adding such token to the existing series of tokens. This auto-regressive operation is represented by the arrow 128.

That is, in a first pass, the pattern completion component 126 generates the added token A₁. In a second pass, the prompt-generating component 126 appends the token A₁ to the end of the existing string of text tokens. The pattern completion component 126 then generates the text token A₂. This process continues until the pattern completion component 126 generates a stop token, which it interprets as a request to stop generating tokens. Upon completion, the pattern completion component 126 provides output information 130 that expresses all of the text tokens added by the pattern completion component 126 in the above-described manner (here, the sequence S₁, S₂, . . . , S_{N} A₁, A₂, . . . , A_{K}). The pattern completion component 126 repeats the above process when new input information is received, including new information reporting the user's current physiological state and/or current emotional state.

The pattern completion component 126 generates each completion based on knowledge of statistical patterns expressed in many other text fragments. That is, the pattern completion component 126 will determine that a string of tokens should be added to the end of an existing string of tokens because it has observed that this pattern is present in many other text fragments.

In some implementations, the pattern completion component 126 is implemented using a machine-trained model 132. The machine-trained model 132 maps a string of input text tokens to a string of output text tokens. The machine-trained model 132 operates based on weights learned by a training system (not shown) in a preceding training process. The training process iteratively adjusts the weights in the course of processing a large corpus of text fragments, with the aim of accurately duplicating the patterns exhibited by those text fragments.

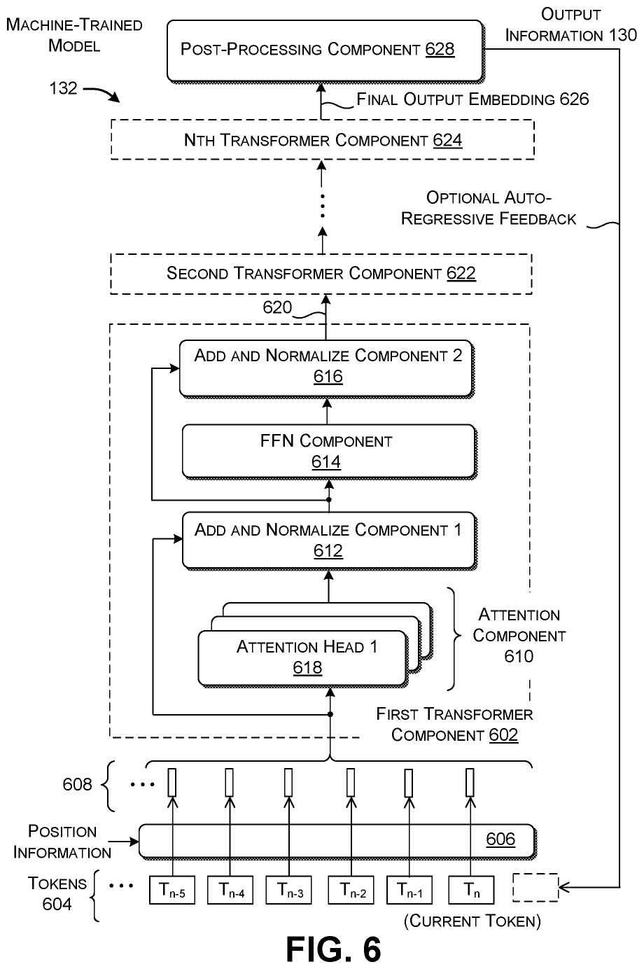

In some cases, the machine-trained model 132 is a transformer-based model. Further details of this type of model are set forth below in connection with FIG. 6. A publicly available transformer-based model for performing pattern completion is the BLOOM model available from HUGGING FACE, INC., of New York, New York, the latest version of which is Version 1.3 released on Jul. 6, 2022. Other implementations of the computing system 102 use of other types of machine-trained models, including fully-connected feed forward neural networks (FFNs), convolutional neural networks (CNNs), recursive neural networks (RNNs), and so on.

The output information 130 includes control instructions for controlling the output system 104. For example, the output information 130 specifies the content to be delivered to the user, e.g., by specified the content of a narrative to be displayed and/or read, the title of a song to be played, the identifiers (e.g., names) of other sounds to be played, the identifiers (e.g., names) of one or more scents to be emitted, and so on. In addition, the output information 130 includes specific commands, values, and/or other control-related data that govern the operation of the output system 104. For example, the output information 130 specifies the characteristics of lighting, including any of a hue, value (lightness), saturation, etc. of the lighting on specified respective scales. In addition, or alternatively, the output information 130 specifies the characteristics of emitted sounds, including any of the volume, bass, treble, balance, etc. of the emitted sounds on specified respective scales. In addition, or alternatively, the output information 130 specifies the characteristics of a scent using any specification system with respect to any scale(s). All of this information originates from the predictions of the pattern completion component 126. For instance, the pattern completion component 126 predicts that a particular song title follows initial text because it has observed that many other text fragments exhibit the same pattern.

With regard to the specific output modality of scent, some specification systems allow a user to express the longevity of a scent and its hedonics. For example, in one illustrative specification system, a “base note” refers to a fragrance that typically lasts more than six hours, a “middle note” refers to a fragrance that typically lasts between three and six hours, and a “high note” refers to a fragrance that typically dissipates within an hour. The scent of Vanilla, for example, often functions as a “base note.” An individual fragrance may combine individual scents having different respective longevities. The hedonics of a scent measures its pleasantness and intensity on specified scales.

The output system 104 delivers multi-sensory content based on the output information 130 using plural output systems. In some implementations, the output system 104 can also directly receive and operate on parts of the input information 106 described above, such as any part of the state information provided by the state-monitoring system 108, as represented in FIG. 1 by a dashed line that connects the input information 106 to the output system 104. The output system 104 includes any of: (a) a visual output system 134; (b) an audio output system 136; (c) an odor output system 138; (c) a lighting system 140; (d) a haptic output system 142; and (e) an HVAC output system (not shown). The visual output system 134 delivers visual content, such as narrative texts, images, and video content. The audio output system 136 delivers audio content, such as spoken narratives, songs, and other sounds. In some implementations, the audio output system 136 incorporates 3D audio effect technology to create an immersive soundscape. One commercially-available technology for providing 3D audio effects is the 360 REALITY AUDIO technology by SONY GROUP CORPORATION of Tokyo, Japan. The odor output system 138 delivers particular scents to the user's environment. One manufacturer of such systems is OVR TECHNOLOGY of Burlington, Vermont. Another is OLORAMA TECHNOLOGY LTD. of Valencia, Spain. The lighting system 140 controls the lighting in the user's environment with respect to physical space (e.g., via overhead lighting). In addition, or alternatively, the headset 226 controls the lighting of a virtual scene based on the output of the lighting system 140. Any other visual output system 134 can likewise adjust one or more control settings based on the output of the lighting system 140. The haptic output system 142 delivers a tactile user experience. One manufacturer of such devices is VR ELECTRONICS LTD. of London, England. The HVAC system (not shown) controls the heating, cooling, and ventilation in a physical environment to complement a simulated scene, e.g., by producing warm balmy conditions to complement a tropical scene. In addition, or alternatively, the HVAC system, and/or some other output system, uses one or more fans to direct breezes to the user's body (e.g., the user's face). The computing system 102 incorporates the winds into the multi-sensory content that it delivers. For instance, in some cases, the computing system 102 uses the fan(s) to simulate the kind of wind to be expected in a particular environment. In other cases, the computing system 102 uses directed air to help guide an intervention. In breathing exercises, for example, the computing system 102 can activate the fan(s) when the user is inhaling and deactivate the fan(s) when the user is exhaling.

One visual system is the image synthesis system 144. The image synthesis system 144 uses a machine-trained model 146 to synthesize an output image based on the output information 130 provided by the pattern completion component 126. For example, assume that part of the output information 130 includes the narrative text: “Sitting on the white sand of a beach, wearing my new sunhat.” The image synthesis system 144 generates an image which depicts a person sitting on a white-sand beach wearing a sun hat. In other cases, the image synthesis system 144 synthesizes multi-modal input information, e.g., by autoregressively mapping a combination of text and image information into new visual content. The concept of “auto-regression” is explained in greater detail below. Further, in some implementations, the image synthesis system 114 produces dynamically-changing visual content. That is, as the stream of output information 130 changes, the image synthesis system 114 dynamically changes its synthesized visual content to reflect the current state of the output information 130.

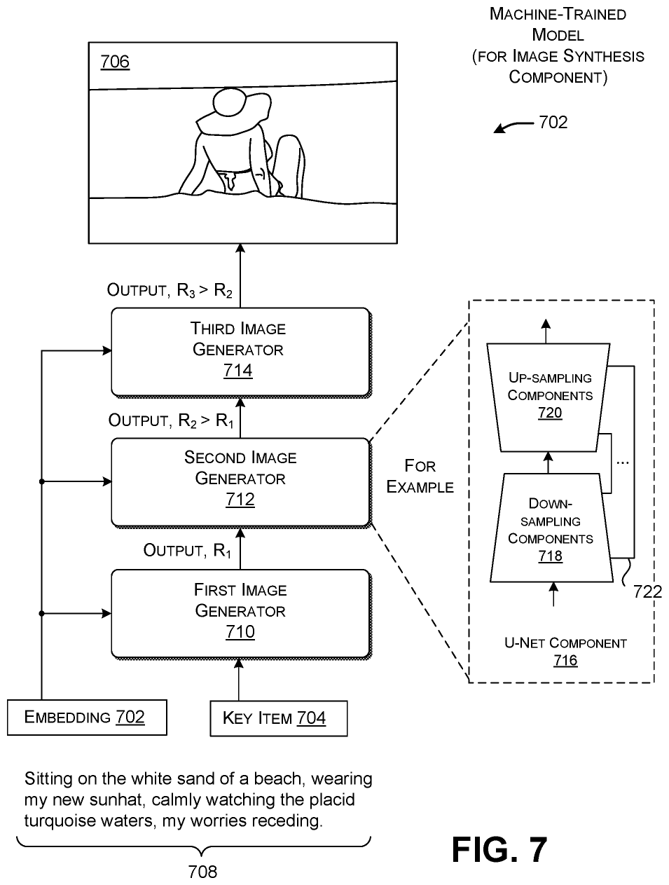

In some cases, the machine-trained model 146 which performs this task is a diffusion model, an example of which is described below in connection with FIG. 7. One publicly available image synthesis engine is provided by the CompVis group at Ludwig Maximilian University of Munich, the latest version of which is Model 2.1 released on Dec. 7, 2022. Another image synthesis system is the DALL-E system provided by OpenAI of San Francisco, California, which is capable of processing a stream composed of text and/or image content. Other types of machine-trained models can achieve the same purpose, including a transformer-based neural network, a CNN, an RNN, and so on. One way to train these other types of models is using a generative adversarial network (GAN).

The visual output system 134 can include one or more other types of visual systems 148. One type of other visual output system chooses from a store of preexisting images and/or videos based on the output information 130.

The computing system 102 provides a flexible tool for serving many different training objectives using a common processing model. The computing system 102 thereby eliminates or reduces the time-intensive and resource-intensive prior practice of developing and maintaining standalone tools that serve specific training objectives. Further, the computing system 102 is user-friendly because a user is able to easily apply it to a specific training problem. To do so, the user need only articulate a goal of training and his or her current emotional state. Further, the computing system 102 provides a sensory experience that is engineered to capture and hold the attention of a user, which increases the chances that training will prove successful.

Further note that the output information 130 is personalized, which further enhances the computing system's ability to capture the attention of the user. This means that the computing system 102 enables the user to construct seed prompt information 122 that contains features that describe the unique characteristics and needs of the user. This seed prompt information 122 causes the pattern completion component 126 to likewise generate personalized output information 130.

FIG. 2 shows an example 202 of an immersive experience produced by the computing system 102 of FIG. 1. In some examples, the user input system 114 enables the user to begin a therapeutic session by entering three items of information via a user interface page 204. As a first item, the user input system 114 invites the user to specify the goal of therapy. In this example, the user specifies that the goal is to “reduce stress.” As a second item, the user input system 114 asks the user to self-report his or her emotional state. In this example, the user specifies that he or she is currently feeling “anxious.” As a third item, the user input system 114 invites the user to describe the type of environment that will be depicted in the multi-sensory content. In this example, the user specifies that he or she wishes the therapy to be framed in context of the themes of “beach” and “sailing.” The user may optionally decline to describe the type of environment, upon which the pattern completion component 126 will choose an environment based on other clues in the prompt information 120. The user may also decline to specify his or her current emotional state, upon which the pattern completion component 126 will generally infer that the user is in need of the therapy requested.

In other implementations, the user input system 114 asks the user to specify additional parameters that will govern the therapeutic session. For instance, the user input system 114 can invite the user to specify what types of media content will be used in the therapeutic session. In addition, or alternatively, the computing system 102 asks the user to specify a technique for structuring the multi-sensory content. For example, assume that the user specifies the established technique of “guided imagery.” In this approach, the computing system 102 presents a series of images with the objective of evoking mental images, memories of sounds and smells, etc., which, in turn, may reactive sensory perception once associated with those memories. Alternatively, or addition, the computing system 102 allows a user to invoke various kinds of mnemonic memory strategies. On such technique is the method of loci that strengthens the ability of a user to remember information in a particular order, guided by a sequence of sensory prompts. For example, assume that the user's objective is to remember the events in their calendar, or facts on which they will later be tested. The computing system 102 creates a story using the memory palace technique with accompanying imagery to weave the events and/or facts into a chronological story conveyed with multi-sensory content.

In some implementations, the user input system 114 allows the user to enter free form answers, without restraint as to what is specified. In other implementations, the user input system 114 allows the user to choose information items from drop-down menus of information items, or the like. In addition, or alternatively, some implementations allow the user to enter information items in explicit spoken form and/or some other form, including non-verbal audible sounds (including sighs, expressive breathing sounds, etc.), sub-vocalization information and silent speech information (e.g., lip movement information), and so on, or any combination thereof. In addition, or alternatively, some implementations allow the user to enter information via gestures, such as by providing a thumbs up or down signal, a facial expression, etc.

The computing system 102 also collects information regarding the current physiological state of the user. FIG. 2 shows, for instance, that one or more vital sign sensing device 206 provide vital sign information (including heart rate, blood pressure, respiratory rate, etc.). A brain activity sensing device 208 collects electroencephalogram information. An electrodermal sensing device 210 collects skin response information. One or more movement sensing device 212 collect body movement information, including any of facial expressions, body posture, type of movement, level of movement, etc. Movement information can indicate the user's level of restlessness, the extent to which the user physically reacts to certain stimuli, etc. An eye state sensing device 214 collects information regarding the user's eyes, including pupil size, direction of gaze, etc. Other implementations collect additional physiological state information. In addition, or alternatively, other implementations omit one or more of the sensing device shown in FIG. 2. In some cases, the computing system 102 provides a workstation which integrates all of the sensing devices. In other cases, the computing system 102 uses a collection of standalone sensing devices.

Although not shown in FIG. 2, some implementations of the computing system 102 collect additional information, such as context-based information (including location and/or current time, etc.).

Assume that the pattern completion component 126 generates prompt information 120 based on the above-described items of information. The pattern completion component 126 maps the prompt information 120 to output information 130. The output system 104 generates multi-sensory content based on the output information 130, and delivers this content via various output devices. By way of overview, the multi-sensory content presents beach-related imagery, beach-related sounds, and beach-related scents, together with a narrative designed to reduce the user's level of stress. The specific nature of this experience depends on the text tokens which make up the prompt information 120. As such, the prompt information 120 serves as a de facto request for a particular kind of experience.

More specifically, the audio output system 136 generates audible content via one or more speakers. For example, the audio output system 136 presents one or more beach-related sounds, such as the sound of lapping waves and/or or the sound of sea birds. In addition, or alternatively, the audio output system 136 presents a beach-themed song, and presents that song to the user. In some cases, the song is instrumental, pursuant to instructions in the prompt information 120 to find a song without lyrics. The song chosen in the example of FIG. 2 includes, for example, a melody in the easy listening genre with tropical stylings (e.g., including the sound of a slide guitar to evoke traditional Hawaiian musical motifs).

In addition, or alternatively, the audio output system 136 reads a narrative generated by the pattern completion component 126. In some examples, the narrative begins with a description of the environment being depicted in images, such as: “I sit relaxing on the beach at sunset. The sun is setting. A cooling breeze gently rustles my beach umbrella. A hear the distant calls of seagulls. I smell the citrus in my drink, which intermingles with the smell of the sea.” The narrative also contains content that more directly attempts to achieve the specified goal of reducing stress, such as: “My heart rate slows down to a soft 59 beats per minutes. I take a deep breath and feel calmer and more composed. Worries fade.” To repeat, the pattern completion component 126 automatically synthesizes this narrative from “scratch” based on the prompt information 120, rather than selecting the narrative from predefined scripts. The narrative therefore reflects patterns of speech in many text fragments, but there is generally no preexisting text that matches the entire delivered narrative.

A visual output system 134 presents visual content in the form of synthesized images and/or video. In addition, or alternatively, the visual output system 134 presents preexisting images and/or video specified in the output information 130.

The visual output system 134 presents the visual content via one or more display devices. As shown in FIG. 2, for example, the visual output system 134 directs a laptop computing device 216 to present an image 218 on its display device 220. The image 218 shows a picture of beach scenery, including a beach umbrella, a sailboat, and a setting sun. In addition, or alternatively, the visual output system 134 presents visual content via a wall display 222. In this example, the wall display 222 shows another image 224 of beach scenery. In some cases, the visual output system 134 transmits digital data describing the visual content to the wall display 222 for display by the wall display 222. Alternatively, the visual output system 134 projects the visual content onto the wall display 222 as images.

In addition, or alternatively, the visual output system 134 presents information using other output devices, such as a virtual or mixed reality headset 226. One commercially available headset of this type is the HOLOLENS headset, produced by MICROSOFT CORPORATION of Redmond, Washington. In some cases, the virtual content is made to appear as if projected onto particular physical surfaces in the user's environment. In addition, or alternatively, the visual output system 134 presents information via an immersive room 228 in which the user can move and explore, sometimes referred to as an immersive cave environment. One provider of immersive environments is VISBOX INC. of Saint Joseph, Illinois. In addition, or alternatively, the visual output system 134 presents visual content via a hologram (not shown), and so on.

The odor output system 138 emits beach-related smells, such as the smell of the sea. The lighting system 140 presents a soft yellow light. The haptic output system 142 simulates the feel of sand touching the skin, the feel of wind impinging the skin, etc. A workflow modifying system 230 controls the user's applications to limit disruptions, e.g., by temporarily silencing alarms, alerts, reminders, and ringing phones. Other environments include yet other types of stimuli, and/or omit one or more forms of stimuli described above.

In some implementations, the multi-sensory content informs the user of his or her progress toward the stated goal of reducing stress. For instance, the visual output system 134 displays the user's current heart rate. In addition, or alternatively, the audio output system 136 presents a narrative which informs the user of his or her progress.

In other cases, the computing system 102 presents imagery, sounds, and narratives appropriate to specific work environments. For example, the computing system 102 displays an operating room environment to train surgeons to cope with the stress of this environment. This is an example of the fact that multi-sensory content need not always be soothing; here, the computing system 102 challenges the user and strengthen his or her ability to successfully cope with the operating room environment.

In other cases, a healthcare provider applies the computing system 102 to the problem of reactivating or bolstering distant memories. To do so, the computing system 102 provides the sounds, images, and smells that accompanied the initial formation of the memories, in the hope of reviving those memories. This technique may allow a user to more fully commit past events to long-term memory. Alternatively, the aim of therapy is to solve the opposite problem: memories are too vivid and intrusive, and are consequently harming the user's health. Therapy assists the user in coping with these memories, e.g., by properly contextualizing them or, in some cases, desensitizing a user towards them.

In yet other cases, the computing system 102 is used for largely entertainment purposes. For instance, the user may commence a session by entering a target environment (e.g., “Medieval England”), a target problem (“slaying a mythical beast”), a target goal (“excitement upon success”), and a current emotional state (“bored”). The computing system 102 thereafter constructs and presents a narrative based on the input information. The narrative evolves based on dynamically changing input conditions, such as the user's current physiological and emotional states.

In all examples, the computing system 102 also enables the user to enter additional descriptive words in the course of a session, such as by typing “I am now ten feet taller” in the course of playing a game. This has the effect of steering the experience to incorporate the new descriptive concepts. In the course of a therapy session of FIG. 2, a user may input “stop showing sailboats.” The computing system 102 adds this information to the prompt information 120 in its current state. The pattern completion component 126 interprets the added information as a request to omit sailboats from the multi-sensory content.

FIG. 3 shows further illustrative details regarding the prompt-generating component 118. As previously described, the prompt-generating component 118 generates prompt information 120. The prompt information 120 has two components: seed prompt information 122 and added information 124.

The seed prompt information 122 includes various items of information. In some examples, the seed prompt information 122 includes: a description of the goal of the training (e.g., “reduce stress”); a description of the desired environment (e.g., “beach”); the user's current self-reported emotional state (e.g., “anxious”); and one or more physiological states. Other implementations include additional information items in the seed prompt information 122 and/or omit one or more of the information items shown in FIG. 3.

The added information 124 includes various updates to the seed prompt information 122. In some examples, the added information 124 includes a description of the selected visual content, audio content, olfactory content, lighting settings, etc. specified by the output information 130. In addition, or alternatively, the added information 124 includes updated measurements from any of the devices that measure the user's physiological state. The computing system 102 may be programmed to supply these updates in the form of prompts on a periodic basis (e.g., every x seconds). Alternatively, or in addition, the output information 130 generated by the pattern completion component 126 includes context-specific instructions to collect updated state information from any sensing device(s). In addition, or alternatively, the added information 124 includes an updated assessment of the user's current emotional state, e.g., as self-reported by the user.

FIG. 4 shows a more detailed example of the seed prompt information 122. A first part 402 of the seed prompt information 122 provides an overview of the training to be provided. It specifies a request for an immersive experience that includes a first-person narrative. The immersive experience uses modulation of light, color, scent, and audio to achieve its effects. The first part 402 also establishes that the user experience is to be guided by the user's current heart rate.

A second part 404 establishes the kind of environment in which the training is to be framed (e.g., “beach”). The second part 404 also establishes the specific goal of the training (e.g., “reduce stress”). The user supplies the information in the second part 404, e.g., by interacting with the user interface page 204. A third part 406 establishes the user's current heart rate, using a current reading provided by a heart rate sensing device. A fourth part 408 establishes the user's current emotional state (e.g., “anxious”), as reported by the user via the user interface page 204. A fifth part 410 specifies the format of control instructions to be used in the output information 130. For instance, the fifth part 410 requests the output information 130 to generate a light intensity setting provided in the range of 0.0 to 1.5. A sixth part 412 specifies the constraints that govern the selection of songs, scents and, narratives. The seed prompt information 122 forms a long sequence of text tokens.

The seed prompt information 122 set forth above directs the pattern completion component 126 to structure the output experience using guided imagery. As noted above, the computing system 102 can invoke many other techniques. For example, the following seed prompt information uses the method of loci described above to help the user enhance his or her memory: “prompt=“Create a first-person story that changes according to the user's heart rate, how they feel”+“and a”+userScenery.text+“scenery.”+“Your goal is to make the person”+userGoal.text+“using guided imagery.”+“Their current heart beats per minute are”+HRtext+“. They currently feel”+userFeeling.text+“.” The seed prompt information continues: “+“Incorporate the following items in your story in order of appearance:”+userMemory.text+“. Use the method of loci based on with this imagery:”+userScenery.text;*/”. (Note that the “+” symbols are a programmatic way to designate the concatenation of strings. In some implementations, the text that is actually fed to the pattern completion component 126 does not include the +” signs.) The prompt-generating component 118 merges this seed prompt information with information collected from the state-monitoring system 108, the user input system 114, and/or the other input system(s) 116. The seed prompt information set forth above specifically directs the pattern completion component 126 to use the method of loci, based on information extracted from specified sources (such as “userMemory.text” and “userScenery.text”).

Now referring to the added information 124, the form and content of this text varies based on the form and content of the seed prompt information 122 fed to the pattern completion component 126. Consider the illustrative case in which the seed prompt information 122 includes the following sequence of text characters: “Prompt: Create an immersive experience in Unity that matches the light color, scent and audio with a first-person story based on my heart rate, how I feel and a beach scenery. Your goal is to help me relax using guided imagery. My current heart beats per minute are 90. I currently feel anxious. Return in one string with this format: {hueHSV}; {saturationHSV}; {valueHSV}; {lightIntensity}; {sound}; {scent}; {firstpersonstory} where hueHSV=0 . . . 1; saturationHSV=0 . . . 1; valueHSV=0 . . . 1; lightIntensity=0 . . . 1.5; sound=titlesongnolyrics; scent=topnotemiddlenotebasenote; story=;”.

Assume that a particular manifestation of the pattern completion component 126 returns the following added information 124: “0.5; 0.5; 0.5; 1; “Soft Waves Crashing” by Kevin Macleod; Lavender, Bergamot, Vanilla; As you close your eyes, you feel a sense of calm wash over you. You are standing on a beach, the sun warming your skin and the sound of the waves crashing against the shore gently lulling you into a state of blissful relaxation. You feel the tension in your body slowly melting away as each breath you take is longer and slower. You can smell the sweet scent of lavender, bergamot, and vanilla in the air. You take a moment to appreciate the beauty of this moment and allow yourself to drift away into a peaceful state.” Note that the actual text consumed and produced by the pattern completion component 126 may omit one or more whitespaces that appear in the above descriptions.

The text “0.5; 0.5; 0.5; 1” in the added information 124 controls the lighting system 140. The pattern completion component 126 produces this information in response to the text “{hueHSV}; {saturationHSV}; {valueHSV}; {lightIntensity}” that appears in the seed prompt information 122, which describes what kind of lighting information is desired, and the text “hueHSV=0 . . . 1; saturationHSV=0 . . . 1; valueHSV=0 . . . 1; lightIntensity=0 . . . 1.5”, which describes the desired formatting of the lighting information.

The text “Soft Wave Crashing” in the added information 124 identifies a particular song to be played by the audio output system 136. The pattern completion component 126 produces this text in response to the part of the seed prompt information 122 that specifies “{sound}”, which identifies that sound is desired, and the text “sound=titlesongnolyrics”, which indicates that the name of a song (without lyrics) is desired.

The text “Lavender, Bergamot, Vanilla” in the added information 124 describes “odor notes” that combine to form a scent presented by the odor output system 138. The pattern completion component 126 produces this text in response to the part of the seed prompt information 122 that specifies “{scent}”, which indicates that a scent is desired, and the text “scent=topnotemiddlenotebasenote”, which describes the desired format to be used to express the scent. Alternatively, or in addition, the seed prompt information 122 can prompt the pattern completion component 126 to generate scents using the formatting information “scent-adjective”. In this format, the pattern completion component 126 is guided to specify a descriptive name of the scent.

The text in the added information 124 that begins “As you close your eyes . . . ” controls a narrative delivered in visual and/or audio form. The pattern completion component 126 presents this text in response to the part of seed prompt information 122 that specifies {firstpersonstory}, which indicates that a first person story is desired. The text “story =;” which prompts the pattern completion component 126 to begin generating a stream of text tokens that compose a story.

Further note that all of the text in the added information 124 is responsive to the contextual information provided in the opening part of the seed prompt information 122. For example, the narrative in the added information 124 reflects the settings set forth in the seed prompt information 122, as do the choice of soundscape, lighting, and scents.

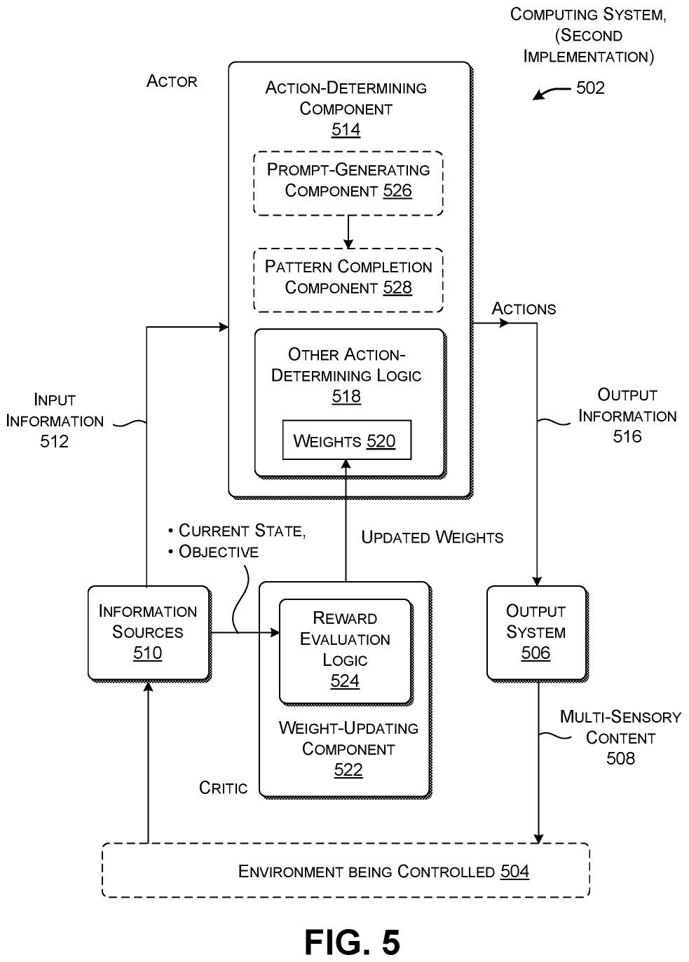

FIG. 5 shows an illustrative computing system 502 that incorporates reinforcement learning. The use of reinforcement learning allows the computing system 502 to modify its behavior over plural sessions based on the user's reaction to content that is delivered. That is, the computing system 502 bolsters the choice of certain stimuli upon discovering that this content advances the stated therapeutic goal. The computing system 502 negatively weights the choice of certain stimuli upon discovering that this content impedes attainment of the goal. In contrast, the pattern completion component 126 used in the computing system 102 of FIG. 1, when operating alone, provides output information 130 that reflects patterns in text provided by many different people; but the pattern completion component 126 does not capture and retain any information that measures the individual user's reaction to the output information 130

An environment 504 refers to an experience delivered by an output system 506. The output system 506 delivers multi-sensory content 508 in the same manner as the output system 104 of FIG. 1. The environment 504 also encompasses the user's reaction to the multi-sensory content 508.

Information sources 510 corresponds to the aggregate of the state-sensing system 108, the user input system 114, and the other input systems 116 shown in FIG. 1. The information sources 510 generate input information 512 which reflects the current state of the environment 504 (including the current physiological and emotional state of the user). In other words, the input information 512 is the counterpart of the input information 106 of FIG. 1.

An action-determining component 514 generates output information 516 based on the input information 512. In some implementations, the action-determining component 514 includes any action-determining logic 518. In some examples, for instance, the action-determining logic 518 includes any type of neural network (an FFN, a CNN, an RNN, a transformer-based network, etc.) for mapping the input information 512 to the output information 516. In this case, the action-determining logic is driven by a set of machine-trained weights 520, which collectively define a policy.

A weight-updating component 522 updates the weights 520 using reinforcement learning. To perform this task, reward evaluation logic 524 determines the extent to which a current state (given by the input information 512) achieves the stated goal of training (also given by the input information 512), in both the short term and the projected long term. For example, assume that the goal is to lower stress. Further assume the user's heart rate and the user's self-reported emotional state are the criteria by which stress is assessed. Further assume that a state of low stress is associated with a heart rate below 65 bpm, and a self-reported assessment of “calm,” “not stressed,” or the equivalent. The reward evaluation logic 524 determines an extent to which the current state departs from the ideal state, and also determines the long-term projected consequence of the current state with respect to the goal of the therapy. The weight-updating component 522 then updates the weights 520 based on this determination. This has the effect of bolstering the kind of stimuli that lowers stress, and penalizing the kind of stimuli that increases stress. In some implementations, the weight-updating component 522 updates the weights using stochastic gradient descent in combination with backpropagation.

In the framework shown in FIG. 5, the action-determining component 518 operates as an actor because it chooses actions which change the environment 504. The reward evaluation logic 524 operates as a critic because it assesses an extent to which a change in the environment 504 advances a stated goal of training. In some implementations (not shown in FIG. 5), the reward evaluation logic 524 may be driven by its own machine-trained model having its own set of weights, separate from the actor's weights 520 in the action-determining logic 518. Here, the weight-updating component 522 also updates the weights of the reward evaluation logic 524 on each iteration. One approach for updating actor and critic weights in this manner is the deep deterministic policy gradient (DDPG) system.

In the above-described manner of operation, the action-determining component 514 relies on just the action-determining logic 518 to generate the output information 516. The action-determining logic 518 is specifically trained to output control instructions in the expected ranges of the individual components of the output system 506.

In a second implementation, the action-determining component 514 relies on the action-determining logic 518 in combination with a prompt-generating component 526 and a pattern completion component 528. The prompt-generating component 526 converts the input information 512 into prompt information in the same manner as the prompt-generating component 118 of FIG. 1. The pattern completion component 528 performs the same role as the pattern completion component 126 of FIG. 1. That is, the pattern completion component 528 maps the prompt information to the output information 516 using a machine-trained model (not shown in FIG. 5). The machine-trained model used by the pattern completion component 528 includes weights that reflect patterns observed in a large corpus of text fragments. But these weights do not reflect whether particular stimuli is advancing or impeding an objective in the present case.

Different implementations combine the analyses of the action-determining logic 518 and the pattern completion component 528 in different respective ways. In one approach, the action-determining logic 518 provides its recommendation, which is based on reinforcement learning, to the prompt-generating component 526 and the pattern completion component 528. The pattern completion component 528 treats this information as just another item of text to take into consideration when generating the output information 516.

In another approach, the action-determining logic 518 operates on the output information 516 generated by the pattern completion component 528, assessing whether the output information 516 will achieve a positive or negative effect. If the latter is the case, the action-determining logic 518 modifies the output information 516, or requests the pattern completion component 528 to generate new output information. In other words, the first approach uses the action-determining logic 518 as a preprocessing component, while the second approach uses the action-determining logic 518 as a post-processing component. Still other ways of integrating these two functionalities are possible.

FIG. 6 shows a one implementation of the machine-trained model 132 (“model” for brevity) used in the pattern completion module 126 of FIG. 1 (and also the machine-trained model used in the pattern completion component 528 of FIG. 5). The model 132 maps initial prompt information 120 to a final output information 130. The model 132 is composed, in part, of a pipeline of transformer components, including a first transformer component 602. FIG. 6 provides details regarding one way to implement the first transformer component 602. Although not specifically illustrated, other transformer components of the model 132 have the same architecture and perform the same functions as the first transformer component 602 (but are governed by separate sets of weights).

The model 132 commences with the receipt of the prompt information 120. The prompt information 120 includes a series of linguistic tokens 604. As used herein, a “token” or “text token” refers to a unit of text having any granularity, such as an individual word, a word fragment produced by byte pair encoding (BPE), a character n-gram, a word fragment identified by the WordPiece algorithm, etc. To facilitate explanation, assume that each token corresponds to a complete word or other unit (e.g., a measurement by a sensing device, or a formatting value).

Next, an embedding component 606 maps the sequence of tokens 604 into respective embedding vectors. For example, the embedding component 606 produces one-hot vectors that describe the tokens, and then uses a machine-trained linear transformation to map the one-hot vectors into the embedding vectors. The embedding component 606 then adds position information to the respective embedding vectors, to produce position-supplemented embedded vectors 608. The position information added to each embedding vector describes the embedding vector's position in the sequence of embedding vectors 608.

The first transformer component 602 operates on the position-supplemented input vectors 608. In some implementations, the first transformer component 602 includes, in order, an attention component 610, a first add-and-normalize component 612, a feed-forward neural network (FFN) component 614, and a second add-and-normalize component 616.

The attention component 610 performs attention analysis using the following equation:

$$attn ( Q , K , V ) = Softmax ( \frac{Q {K}^{T}}{\sqrt{d}} ) V . \qquad ( 1 )$$

The attention component 610 produces query information Q by multiplying the position-supplemented embedded vectors 608 (or, in some applications, just a last position-supplemented embedding vector associated with a last-received token) by a query weighting matrix W^{Q}. Similarly, the attention component 416 produces key information K and value information V by multiplying the position-supplemented embedding vectors by a key weighting matrix WK and a value weighting matrix WV, respectively. To execute Equation (1), the attention component 416 takes the dot product of Q with the transpose of K, and then divides the dot product by a scaling factor \sqrt{d}, to produce a scaled result The symbol d represents the dimensionality of Q and K. The attention component 610 takes the Softmax (normalized exponential function) of the scaled result, and then multiplies the result of the Softmax operation by V, to produce attention output information. More generally stated, the attention component 610 determines how much emphasis should be placed on parts of the input information when interpreting other parts of the input information. In some cases, the attention component 610 is said to perform masked attention insofar as the attention component 610 masks output token information that, at any given time, has not yet been determined. Background information regarding the general concept of attention is provided in Vaswani, et al., “Attention Is All You Need,” in 31st Conference on Neural Information Processing Systems (NIPS 2017), 2017, 11 pages.

Note that FIG. 6 shows that the attention component 610 is composed of plural attention heads, including a representative attention head 618. Each attention head performs the computations specified by Equation (1), but with respect to a particular representational subspace that is different than the subspaces of the other attention heads. To accomplish this operation, the attention heads perform the computations described above using different respective sets of query, key, and value weight matrices. Although not shown, the attention component 610 concatenates the output results of the attention component's separate attention heads, and then multiplies the results of this concatenation by another weight matrix W^{O}.

The add-and-normalize component 612 includes a residual connection that combines (e.g., sums) input information fed to the attention component 610 with the output information generated by the attention component 610. The add-and-normalize component 612 then normalizes the output information generated by of the residual connection, e.g., by normalizing values in the output information based on the mean and standard deviation of those values. The other add-and-normalize component 616 performs the same functions as the first-mentioned add-and-normalize component 612.

The FFN component 614 transforms input information to output information using a feed-forward neural network having any number of layers. In some implementations, the FFN component 614 is a two-layer network that performs its function using the following equation:

$$FNN ( x ) = max ( 0 , {xW}_{f n n 1} + {b}_{1} ) {W}_{f n n 2} + {b}_{2} . \qquad ( 2 )$$

The symbols W_{fnn1} and W_{fnn2} refer to two weight matrices used by the FFN component 614, having reciprocal shapes of (d, d_{fnn}) and (d_{fnn}, d), respectively. The symbols b₁ and b₂ represent bias values.

The first transformer component 602 produces an output embedding 620. A series of other transformer components (622, . . . , 624) perform the same functions as the first transformer component 602, each operating on an output embedding produced by its immediately preceding transformer component. Each transformer component uses its own level-specific set of machine-trained weights. The final transformer component 624 in the model 132 produces a final output embedding 626.

A post-processing component 628 performs post-processing operations on the final output embedding 626, to produce the final output information 130. In one case, for instance, the post-processing component 628 performs a machine-trained linear transformation on the final output embedding 626, and processes the result of this transformation using a Softmax component (not shown).

In some implementations, the model 132 operates in an auto-regressive manner. To operate in this way, the post-processing component 628 uses the Softmax operation to predict a next token (or, in some cases, a set of the most probable next tokens). The model 132 then appends the next token to the end of the sequence of input tokens 604, to provide an updated sequence of tokens. In a next pass, the model 132 processes the updated sequence of tokens to generate a next output token. The model 132 repeats the above process until it generates a specified stop token.

In one implementation, a training system (not shown) trains the weights of the model 132 based on a large corpus of text fragments. These text fragments pertain to different domains of knowledge, and do not target any specific domain. In some cases, the training system optionally fine-tunes the weights based on a corpus of text fragments that are particularly appropriate to the kinds of training provided by the computing system 102. For example, this implementation fine-tunes the weights based on a corpus of documents pertaining to various applicable medical-related fields, psychology-related fields, and sociology-related fields.

FIG. 7 shows an example of the machine-trained model 146 (“model” for brevity) used in the image synthesis system 144 of FIG. 1. FIG. 7 specifically shows the case in which the model 146 is a diffusion model that maps an embedding 702 and a key item 704 to an image 706. Assume that any encoder-type machine-trained model (not shown) has produced the embedding 702 based on a text item 708, as primed by a randomly-generated instance of noise information. The key item 704 corresponds to the randomly-generated instance of noise information. The text item 708, in turn, is produced, at least in part, by the pattern-completion component 126. In other words, the text item 708 corresponds to part of the output information 130 (with reference to FIG. 1).

In some implementations, the model 146 successively transforms the key item 704 (which represents a sample of noise) into the image 706, as guided by the embedding 702, using a series of image generators (710, 712, 714). The first image generator 710 produces image information having a resolution of R₁. The second image generator 712 produces image information having a resolution of R₂, where R₂>R₁. The third image generator 714 produces image information having a resolution of R₃, where R₃>R₂, and so on. In some implementations, the diffusion model 146 implements each image generator using a U-Net component. For instance, with respect to the presentative second image generator 712, a U-Net component 716 includes a series of down-sampling components 718 followed by a series of up-sampling components 720. Each down-sampling component or up-sampling component itself includes any combination of sub-components, including any of a convolutional component, a feed-forward component, a residual connection, an attention component, etc. Skip connections 722 couple down-sampling and up-sampling components that perform processing with respect to the same resolution level.

More specifically, the input to the machine-trained model 146 may take the form of prompt information generated by the pattern completion component 126. For example, in one case, the prompt information generated by the pattern completion component 126 takes the form of: “string prompt=+timer+“equirectangular scenery based on the following story:”+movingWindowInput+“.”;”. The prefatory information “equirectangular scenery based on the following story:” prompts the machine-trained model 702 to provide imagery in the form of a spherical/skybox image, instead of 2D imagery.

The machine-trained model 146 dynamically changes its imagery based on changes in the variable “movingWindowInput”. The text associated with the variable “movingWindowInput” changes, in turn, based on changes in the prompt information 120 fed to the pattern completion component 126. Consider the case in which the user's stress level spikes for some reason. The prompt information 120 fed to the pattern completion component 126 will change to reflect updated state information that expresses the user's spike in stress. This changes trickles down to affect the output information 130, and then to affect the imagery generated by the image synthesis system 144.

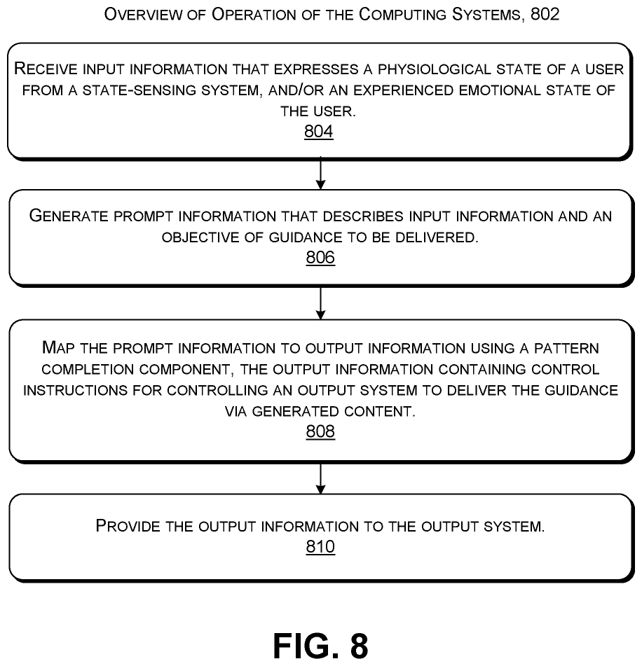

FIG. 8 shows a first process 802 that provides an overview of the computing system 102 of FIG. 1. Although explained in the context of the computing system 102 of FIG. 1, the process 802 also applies to some implementations of the computing system 502 of FIG. 5. More generally, the process 802 is expressed as a series of operations performed in a particular order. But the order of these operations is merely representative, and the operations are capable of being varied in other implementations. Further, any two or more operations described below can be performed in a parallel manner. In one implementation, the blocks shown in the flowcharts that pertain to processing-related functions are implemented by the hardware logic circuitry described in connection with FIGS. 10 and 11, which, in turn, is implemented by one or more processors, a computer-readable storage medium, etc.

In block 804, the computing system 102 receives input information (e.g., input information 106) that expresses a physiological state of a user from a state-sensing system (e.g., the state-sensing system 108), and/or an experienced emotional state of a user (e.g., from the user input system 114). In block 806, the computing system 102 generates prompt information (e.g., prompt information 120) that describes input information and an objective of guidance to be delivered; In block 808, the computing system 102 maps the prompt information to output information (e.g., output information 130) using a pattern completion component (e.g., the pattern completion component 126), the output information containing control instructions for controlling an output system (e.g., the output system 104) to deliver the guidance via generated content. In block 810, the computing system 102 provides the output information to the output system.

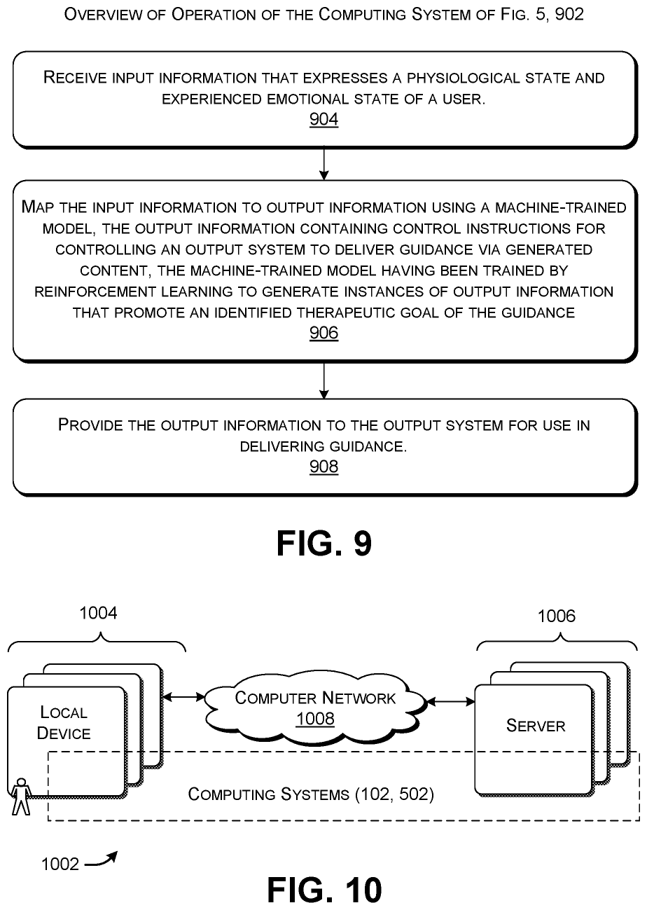

FIG. 9 shows a second process 902 that provides an overview of the computing system 502 of FIG. 5. The same generalization provided for FIG. 8 applies to the process 902 of FIG. 9. The flow shown in FIG. 9 is one flow among other possible flows.

In block 904, the computing system 502 receives input information (e.g., input information 512) that expresses a physiological state of a user from a state-sensing system (e.g., the state-sensing system 108) and experienced emotional state of a user (e.g., obtained from the user input system 114). In block 906, the computing system 502 maps the input information to output information (e.g., the output information 516) using a machine-trained model (e.g., using the action-determining logic 518), the output information containing control instructions for controlling an output system (e.g., the output system 506) to deliver guidance via generated content (e.g., the multi-sensory content 508). The machine-trained model is trained by reinforcement learning to generate instances of output information that promote an identified therapeutic goal of the guidance. In block 908, the computing system provides the output information to the output system for use in delivering guidance.

FIG. 10 shows computing equipment 1002 that, in some implementations, is used to implement the computing system 102 of FIG. 1 or the computing system 502 of FIG. 5. The computing equipment 1002 includes a set of local devices 1004 coupled to a set of servers 1006 via a computer network 1008. Each local device corresponds to any type of computing device, including any of a desktop computing device, a laptop computing device, a handheld computing device of any type (e.g., a smartphone or a tablet-type computing device), a mixed reality device, an intelligent appliance, a wearable computing device (e.g., a smart watch), an Internet-of-Things (IoT) device, a gaming system, an immersive “cave,” a media device, a vehicle-borne computing system, any type of robot computing system, a computing system in a manufacturing system, etc. In some implementations, the computer network 1008 is implemented as a local area network, a wide area network (e.g., the Internet), one or more point-to-point links, or any combination thereof.

The dashed-line box in FIG. 10 indicates that the functionality of the computing systems (102, 502) is capable of being spread across the local devices 1004 and/or the servers 1006 in any manner. For instance, in some cases, each local commuting device, or a group of affiliated local computing devices, implements the entirety the computing systems (102, 502). In other implementations, one or data-processing operations are delegated to an online resource provided by the servers 1006. For example, in some implementations, the pattern completion components (126, 528) are implemented by the servers 1006.

FIG. 11 shows a computing system 1102 that, in some implementations, is used to implement any aspect of the mechanisms set forth in the above-described figures. For instance, in some implementations, the type of computing system 1102 shown in FIG. 11 is used to implement any local computing device or any server shown in FIG. 10. In all cases, the computing system 1102 represents a physical and tangible processing mechanism.

The computing system 1102 includes a processing system 1104 including one or more processors. The processor(s) include one or more Central Processing Units (CPUs), and/or one or more Graphics Processing Units (GPUs), and/or one or more Application Specific Integrated Circuits (ASICs), and/or one or more Neural Processing Units (NPUs), etc. More generally, any processor corresponds to a general-purpose processing unit or an application-specific processor unit.

The computing system 1102 also includes computer-readable storage media 1106, corresponding to one or more computer-readable media hardware units. The computer-readable storage media 1106 retains any kind of information 1108, such as machine-readable instructions, settings, model weights, and/or other data. In some implementations, the computer-readable storage media 1106 includes one or more solid-state devices, one or more magnetic hard disks, one or more optical disks, magnetic tape, etc. Any instance of the computer-readable storage media 1106 uses any technology for storing and retrieving information. Further, any instance of the computer-readable storage media 1106 represents a fixed or removable unit of the computing system 1102. Further, any instance of the computer-readable storage media 1106 provides volatile and/or non-volatile retention of information.

More generally, any of the storage resources described herein, or any combination of the storage resources, is to be regarded as a computer-readable medium. In many cases, a computer-readable medium represents some form of physical and tangible entity. The term computer-readable medium also encompasses propagated signals, e.g., transmitted or received via a physical conduit and/or air or other wireless medium. However, the specific term “computer-readable storage medium” or “storage device” expressly excludes propagated signals per se in transit, while including all other forms of computer-readable media; a computer-readable storage medium or storage device is “non-transitory” in this regard.

The computing system 1102 utilizes any instance of the computer-readable storage media 1106 in different ways. For example, in some implementations, any instance of the computer-readable storage media 1106 represents a hardware memory unit (such as random access memory (RAM)) for storing information during execution of a program by the computing system 1102, and/or a hardware storage unit (such as a hard disk) for retaining/archiving information on a more permanent basis. In the latter case, the computing system 1102 also includes one or more drive mechanisms 1110 (such as a hard drive mechanism) for storing and retrieving information from an instance of the computer-readable storage media 1106.

In some implementations, the computing system 1102 performs any of the functions described above when the processing system 1104 executes computer-readable instructions stored in any instance of the computer-readable storage media 1106. For instance, in some implementations, the computing system 1102 carries out computer-readable instructions to perform each block of the processes described in with reference to FIGS. 9 and 10. FIG. 11 generally indicates that hardware logic circuitry 1112 includes any combination of the processing system 1104 and the computer-readable storage media 1106.

In addition, or alternatively, the processing system 1104 includes one or more other configurable logic units that perform operations using a collection of logic gates. For instance, in some implementations, the processing system 1104 includes a fixed configuration of hardware logic gates, e.g., that are created and set at the time of manufacture, and thereafter unalterable. In addition, or alternatively, the processing system 1104 includes a collection of programmable hardware logic gates that are set to perform different application-specific tasks. The latter category of devices includes Programmable Array Logic Devices (PALs), Generic Array Logic Devices (GALs), Complex Programmable Logic Devices (CPLDs), Field-Programmable Gate Arrays (FPGAs), etc. In these implementations, the processing system 1104 effectively incorporates a storage device that stores computer-readable instructions, insofar as the configurable logic units are configured to execute the instructions and therefore embody or store these instructions.

In some cases (e.g., in the case in which the computing system 1102 represents a user computing device), the computing system 1102 also includes an input/output interface 1114 for receiving various inputs (via input devices 1116), and for providing various outputs (via output devices 1118). Illustrative input devices include a keyboard device, a mouse input device, a touchscreen input device, a digitizing pad, one or more static image cameras, one or more video cameras, one or more depth camera systems, one or more microphones, a voice recognition mechanism, any position-determining devices (e.g., GPS devices), any movement detection mechanisms (e.g., accelerometers and/or gyroscopes), etc. In some implementations, one particular output mechanism includes a display device 1120 and an associated graphical user interface presentation (GUI) 1122. The display device 1120 corresponds to a liquid crystal display device, a light-emitting diode display (LED) device, a cathode ray tube device, a projection mechanism, etc. Other output devices include a printer, one or more speakers, a haptic output mechanism, an archival mechanism (for storing output information), etc. In some implementations, the computing system 1102 also includes one or more network interfaces 1124 for exchanging data with other devices via one or more communication conduits 1126. One or more communication buses 1128 communicatively couple the above-described units together.

The communication conduit(s) 1126 is capable of being implemented in any manner, e.g., by a local area computer network, a wide area computer network (e.g., the Internet), point-to-point connections, or any combination thereof. The communication conduit(s) 1126 include any combination of hardwired links, wireless links, routers, gateway functionality, name servers, etc., governed by any protocol or combination of protocols.

FIG. 11 shows the computing system 1102 as being composed of a discrete collection of separate units. In some cases, the collection of units corresponds to discrete hardware units provided in a computing device chassis having any form factor. FIG. 11 shows illustrative form factors in its bottom portion. In other cases, the computing system 1102 includes a hardware logic unit that integrates the functions of two or more of the units shown in FIG. 1. For instance, in some implementations, the computing system 1102 includes a system on a chip (SoC or SOC), corresponding to an integrated circuit that combines the functions of two or more of the units shown in FIG. 11.

The following summary provides a set of illustrative examples of the technology set forth herein.

(A1) According to a first aspect, a computer-implemented method (e.g., 802) is described for providing content. The method includes: receiving (e.g., 804) input information (e.g., 106) that expresses a physiological state of a user obtained from a state-sensing system, and/or an experienced emotional state of the user; generating (e.g., 806) prompt information (e.g., 120) that describes the input information and an objective of guidance to be delivered; mapping (e.g., 808) the prompt information to output information (e.g., 130) using a pattern completion component (e.g., 126), the output information containing control instructions for controlling an output system (e.g., 104) to deliver the guidance via generated content; and providing (810) the output information to the output system.

(A2) According to some implementations of the method of A1, the physiological state of the user expresses: (a) a vital sign; or (b) electrodermal activity; or (c) body movement; or (d) an eye-related characteristic; or (e) a voice-related characteristic; or (f) any combination thereof.

(A3) According to some implementations of the methods of A1 or A2, the emotional state is self-reported by the user.

(A4) According to some implementations of any individual method of the methods of A1-A3, the objective expressed in the prompt information is a therapeutic goal of the guidance.

(A5) According to some implementations of the method of A4, the therapeutic goal is: (a) reduction of stress; or (b) meditation; or (c) inducement of sleep; or (d) inducement of attentiveness; or (e) control of a specified emotion or compulsion; or (f) management of memory; or (g) ability to complete a task within a specified environment; or (h) enhancement productivity; or (i) any combination thereof.

(A6) According to some implementations of any individual method of the methods of A1-A5, wherein the prompt information also describes a selected environment.

(A7) According to some implementations of any individual method of the methods of A1-A6, the prompt information is a series of input text tokens, the pattern completion component is a machine-trained model, and the output information is a series of output text tokens.

(A8) According to some implementations of the method of A7, the machine-trained model a transformer-based machine-trained neural network.

(A9) According to some implementations of the method of A7, at least some of the input text tokens describe a type of output modality to use, and at least some of the input text tokens describe a format of control information to be provided in the output text tokens.

(A10) According to some implementations of the method of A7, the input information and/or the output information is further processed by another machine-trained model, the other machine-trained model being trained by reinforcement learning to promote the objective of the guidance.

(A11) According to some implementations of any individual method of the methods of A1-A10, the output system includes a machine-trained model for mapping the output information to visual content.

(A12) According to some implementations of any individual method of the methods of A1-A11 the content is multi-sensory content.

(A13) According to some implementations of any individual method of the methods of A1-A12, the output system includes: (a) an audio output system for delivering audio content; or (b) a visual output system for delivering visual content; or (c) a lighting system for controlling lighting; or (d) an odor output system for delivering scents; or (e) a haptic output system for delivering a tactile experience; or an (f) HVAC system for controlling heating, cooling, and/or ventilation, or (g) a workflow-modifying system for controlling workflow of the user; or (h) any combination thereof.

(A14) According to some implementations of any individual method of the methods of A1-A13, the method further includes updating the prompt information to include aspects of the output information and updated state information, to provide updated prompt information, and mapping the updated prompt information to updated output information using the pattern completion component.

(B1) According a second aspect, another computer-implemented method (e.g., 902) is described for providing content. The method includes: receiving (e.g., 904) input information (512) that expresses a physiological state of a user obtained from a state-sensing system, and an experienced emotional state of the user; mapping (906) the input information to output information (e.g., 516) using a machine-trained model (e.g., 518), the output information containing control instructions for controlling an output system (e.g., 506) to deliver guidance via generated content; and providing (e.g., 908) the output information to the output system for use in delivering guidance. The machine-trained model is trained by reinforcement learning to generate instances of output information that promote an identified therapeutic goal of the guidance.

(B2) According to some implementations of the method of B1, the operations further include generating prompt information that describes the input information and the therapeutic goal of guidance to be delivered. The output information is also produced by mapping the prompt information to candidate output information using a pattern completion component.

In yet another aspect, some implementations of the technology described herein include a computing system (e.g., the computing system 1102) that includes a processing system (e.g., the processing system 1104) having a processor. The computing system also includes a storage device (e.g., the computer-readable storage media 1106) for storing computer-readable instructions (e.g., information 1108). The processing system executes the computer-readable instructions to perform any of the methods described herein (e.g., any individual method of the methods of A1-A14, B1, or B2).

In yet another aspect, some implementations of the technology described herein include a computer-readable storage medium (e.g., the computer-readable storage media 1106) for storing computer-readable instructions (e.g., the information 1108). A processing system (e.g., the processing system 1104) executes the computer-readable instructions to perform any of the operations described herein (e.g., the operation in any individual method of the methods of A1-A14, B1, or B2).

More generally stated, any of the individual elements and steps described herein are combinable into any logically consistent permutation or subset. Further, any such combination is capable of being be manifested as a method, device, system, computer-readable storage medium, data structure, article of manufacture, graphical user interface presentation, etc. The technology is also expressible as a series of means-plus-format elements in the claims, although this format should not be considered to be invoked unless the phrase “means for” is explicitly used in the claims.

As to terminology used in this description, the phrase “configured to” encompasses various physical and tangible mechanisms for performing an identified operation. The mechanisms are configurable to perform an operation using the hardware logic circuitry 1112 of FIG. 11. The term “logic” likewise encompasses various physical and tangible mechanisms for performing a task. For instance, each processing-related operation illustrated in the flowcharts of FIGS. 11 and 12 corresponds to a logic component for performing that operation.

This description may have identified one or more features as optional. This type of statement is not to be interpreted as an exhaustive indication of features that are to be considered optional; generally, any feature is to be considered as optional, although not explicitly identified in the text, unless otherwise noted. Further, any mention of a single entity is not intended to preclude the use of plural such entities; similarly, a description of plural entities in the specification is not intended to preclude the use of a single entity. As such, a statement that an apparatus or method has a feature X does not preclude the possibility that it has additional features. Further, any features described as alternative ways of carrying out identified functions or implementing identified mechanisms are also combinable together in any combination, unless otherwise noted.

In terms of specific terminology, the term “plurality” or “plural” or the plural form of any term (without explicit use of “plurality” or “plural”) refers to two or more items, and does not necessarily imply “all” items of a particular kind, unless otherwise explicitly specified. The term “at least one of” refers to one or more items; reference to a single item, without explicit recitation of “at least one of” or the like, is not intended to preclude the inclusion of plural items, unless otherwise noted. Further, the descriptors “first,” “second,” “third,” etc. are used to distinguish among different items, and do not imply an ordering among items, unless otherwise noted. The phrase “A and/or B” means A, or B, or A and B. The phrase “any combination thereof” refers to any combination of two or more elements in a list of elements. Further, the terms “comprising,” “including,” and “having” are open-ended terms that are used to identify at least one part of a larger whole, but not necessarily all parts of the whole. A “set” includes zero members, one member, or more than one member. Finally, the terms “exemplary” or “illustrative” refer to one implementation among potentially many implementations.

In closing, the functionality described herein is capable of employing various mechanisms to ensure that any user data is handled in a manner that conforms to applicable laws, social norms, and the expectations and preferences of individual users. For example, the functionality is configurable to allow a user to expressly opt in to (and then expressly opt out of) the provisions of the functionality. The functionality is also configurable to provide suitable security mechanisms to ensure the privacy of the user data (such as data-sanitizing mechanisms, encryption mechanisms, and/or password-protection mechanisms).

Further, the description may have set forth various concepts in the context of illustrative challenges or problems. This manner of explanation is not intended to suggest that others have appreciated and/or articulated the challenges or problems in the manner specified herein. Further, this manner of explanation is not intended to suggest that the subject matter recited in the claims is limited to solving the identified challenges or problems; that is, the subject matter in the claims may be applied in the context of challenges or problems other than those described herein.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method for providing content, comprising:
receiving input information that expresses a physiological state of a user obtained from a state-sensing system, and/or an experienced emotional state of the user;
generating prompt information that describes the input information and an objective of guidance to be delivered;
mapping the prompt information to output information using a pattern completion component, the output information containing control instructions for controlling an output system to deliver the guidance via generated content; and
providing the output information to the output system.

2. The method of claim 1, wherein the physiological state of the user expresses: (a) a vital sign; or (b) electrodermal activity; or (c) body movement; or (d) an eye-related characteristic; or (e) a voice-related characteristic; or (f) any combination thereof.

3. The method of claim 1, wherein the emotional state is self-reported by the user.

4. The method of claim 1, wherein the objective expressed in the prompt information is a therapeutic goal of the guidance.

5. The method of claim 4, wherein the therapeutic goal is:
(a) reduction of stress; or
(b) meditation; or
(c) inducement of sleep; or
(d) inducement of attentiveness; or
(e) control of a specified emotion or compulsion; or
(f) management of memory; or
(g) ability to complete a task within a specified environment;
(h) enhancement of productivity; or
(i) any combination thereof.

6. The method of claim 1, wherein the prompt information also describes a selected environment.

7. The method of claim 1,
wherein the prompt information is a series of input text tokens,
wherein the pattern completion component is a machine-trained model, and
wherein the output information is a series of output text tokens.

8. The method of claim 7, wherein the machine-trained model a transformer-based machine-trained neural network.

9. The method of claim 7, wherein at least some of the input text tokens describe a type of output modality to use, and at least some of the input text tokens describe a format of control information to be provided in the output text tokens.

10. The method of claim 7, wherein the input information and/or the output information is further processed by another machine-trained model, the other machine-trained model being trained by reinforcement learning to promote the objective of the guidance.

11. The method of claim 1, wherein the output system includes a machine-trained model for mapping the output information to visual content.

12. The method of claim 1, wherein the content is multi-sensory content.

13. The method of claim 1, wherein the output system includes: (a) an audio output system for delivering audio content; or (b) a visual output system for delivering visual content; or (c) a lighting system for controlling lighting; or (d) an odor output system for delivering scents; or (e) a haptic output system for delivering a tactile experience; or (f) an HVAC system for controlling heating, cooling, and/or ventilation, or (g) a workflow-modifying system for controlling workflow of the user; or (h) any combination thereof.

14. The method of claim 1, further including updating the prompt information to include aspects of the output information and updated state information, to provide updated prompt information, and mapping the updated prompt information to updated output information using the pattern completion component.

15. A computing system for providing content;
a store for storing computer-readable instructions;
a processing system for executing the computer-readable instructions to perform operations that include:
receiving input information that expresses a physiological state of a user from a state-sensing system, and an experienced emotional state of the user;
mapping the input information to output information using a machine-trained model, the output information containing control instructions for controlling an output system to deliver guidance via generated content; and
providing the output information to the output system for use in delivering guidance,
the machine-trained model having been trained by reinforcement learning to generate instances of output information that promote an identified therapeutic goal of the guidance.

16. The computing system of claim 15, wherein the output system includes another machine-trained model for mapping the output information to visual content.

17. The computing system of claim 15, wherein the output system includes: (a) an audio output system for delivering audio content; or (b) a visual output system for delivering visual content; or (c) a lighting system for modifying lighting; or (d) an odor output system for delivering scents; or (e) a haptic output system for delivering a tactile experience; or an (f) HVAC system for controlling heating, cooling, and/or ventilation (g) a workflow-modifying system; or (h) any combination thereof.

18. The computing system of claim 14,
wherein the operations further include generating prompt information that describes the input information and the therapeutic goal of guidance to be delivered, and
wherein the output information is also produced by mapping the prompt information to candidate output information using a pattern completion component.

19. The computing system of claim 18,
wherein the prompt information is a series of input text tokens,
wherein the pattern completion component is a machine-trained pattern completion model, and
wherein the candidate output information is a series of output text tokens.

20. A computer-readable storage medium for storing computer-readable instructions, a processing system executing the computer-readable instructions to perform operations, the operations comprising:
receiving input information that expresses that expresses a current state of a user;
generating prompt information that describes an objective of guidance to be delivered, the input information, and a description of a selected environment; and
mapping the prompt information to output information using a machine-trained pattern completion model, the output information containing control instructions for controlling an output system to deliver the guidance via generated multi-sensory content,
wherein the input information and/or the output information is further processed by another machine-trained model, the other machine-trained model being trained by reinforcement learning to promote the objective of the guidance.
